(19) United States
(12) Patent Application Publication
(10) Pub. No.: US 2001/0051754 A1
(43) Pub. Date: Dec. 13, 2001

(54) PLURAL STAGE TOLUENE DISPROPORTIONATION PROCESS MINIMIZING ETHYLBENZENE

(76) Inventors: DARIA N. LISSY, GLEN MILLS, PA (US); SANJAY B. SHARMA, BURR RIDGE, IL (US); DAVID S. SHIHABI, VOORHEES, NJ (US)

Correspondence Address: EXXONMOBIL CHEMICAL COMPANY, P O BOX 2149, BAYTOWN, TX, 775222149

(21) Appl. No.: 09/366,477

(22) Filed: Aug. 3, 1999

(51) Int. Cl. C07C 2/66; C07C 5/22

(52) U.S. Cl. 585/319; 585/411; 585/419; 585/470; 585/475; 585/486; 585/489

(57) ABSTRACT

A process for toluene disproportionation which obtains high xylene yields while minimizing ethylbenzene production employs a dual catalyst bed. The first bed employs an acid zeolite, e.g., ZSM-5 which disproportionates toluene and the downstream second bed uses an acid zeolite having hydrogenation-dehydrogenation activity, e.g., PtZSM-5, to selectively eliminate ethylbenzene.

### FIELD OF THE INVENTION

The present invention relates to a process for toluene disproportionation (TDP) which produces xylenes and minimizes ethylbenzene production.

### BACKGROUND OF THE INVENTION

The present invention is directed to a process for shape-selective hydrocarbon conversions such as the regioselective conversion of toluene to xylenes with minimal ethylbenzene production, using at least two stages. The process provides higher xylene yields compared to corresponding single bed processes.

The term shape selective catalysis describes unexpected catalytic selectivities in zeolites. The principles behind shape selective catalysis have been reviewed extensively, e.g., by N. Y. Chen, W. E. Garwood and F. G. Dwyer, “Shape Selective Catalysis in Industrial Applications,” 36, Marcel Dekker, Inc. (1989). Within a zeolite pore, hydrocarbon conversion reactions such as paraffin isomerization, olefin skeletal or double bond isomerization, oligomerization and aromatic disproportionation, alkylation or transalkylation reactions are governed by constraints imposed by the channel size. Reactant selectivity occurs when a fraction of the feedstock is too large to enter the zeolite pores to react; while product selectivity occurs when some of the products cannot leave the zeolite channels. Product distributions can also be altered by transition state selectivity in which certain reactions cannot occur because the reaction transition state is too large to form within the zeolite pores or cages. Another type of selectivity results from configurational diffusion where the dimensions of the molecule approach that of the zeolite pore system. A small change in dimension of the molecule or the zeolite pore can result in large diffusion changes leading to different product distributions. This type of shape selective catalysis is demonstrated, for example, in toluene selective disproportionation to p-xylene.

Para-xylene is a very valuable commercial product useful in the production of polyester fibers. The catalytic production of para-xylene has received much attention in the scientific community and various methods for increasing catalyst para-selectivity have been described.

The synthesis of para-xylene is typically performed by methylation of toluene over a catalyst under conversion conditions. Examples are the reaction of toluene with methanol as described by Chen et al., J. Amer. Chem. Soc. 1979, 101, 6783, and toluene disproportionation, as described by Pines in “The Chemistry of Catalytic Hydrocarbon Conversions,” Academic Press, N.Y., 1981, p. 72. Such methods typically result in the production of a mixture including para-xylene, ortho-xylene, and meta-xylene. Depending upon the para-selectivity of the catalyst and the reaction conditions, different percentages of para-xylenes are obtained. The yield, i.e., the amount of feedstock actually converted to xylene, is also affected by the catalyst and the reaction conditions.

One known method for increasing para-selectivity of zeolite catalysts is to modify the catalyst by treatment with “selectivating agents.” Modification methods have been suggested wherein the catalyst is modified by treatment prior to use to provide a silica coating. For example, U.S. Pat. Nos. 4,477,583 and 4,127,616 disclose methods wherein a catalyst is contacted at ambient conditions with a modifying compound such as phenylmethyl silicone in a hydrocarbon solvent or an aqueous emulsion, followed by calcination. Such modification procedures have been successful in obtaining para-selectivity of greater than about 90% but with commercially unacceptable toluene conversions of only about 10%, resulting in a yield of not greater than about 9%, i.e., 10% times 90%. Such processes also produce significant quantities of ortho-xylene and meta-xylene thereby necessitating expensive separation processes in order to separate the para-xylene from the other isomers.

U.S. Pat. No. 5,498,814 to Chang et al. discloses regioselective disproportionation of toluene to para-xylene wherein hydrocarbon is passed over a catalytic molecular sieve such as ZSM-5 which is trim selectivated with a reaction stream comprising toluene and a silicon-containing high efficiency p-xylene selectivating agent under toluene conversion conditions. The toluene disproportionation process can attain single-pass para-xylene product over 95% coupled with a toluene conversion of at least 15%.

U.S. Pat. No. 5,516,956 to Abichandani et al. discloses isomerizing a mixture of ethylbenzene and xylene using a two component catalyst system to convert the ethylbenzene to compounds that may be removed from an aromatic hydrocarbon stream and to produce a product stream with para-xylene concentration equal to the equilibrium ratio of the para-isomer. The first catalyst comprises silica-bound intermediate pore size zeolite effective for ethylbenzene conversion while the second catalyst comprises small crystal size intermediate pore size zeolite effective to catalyze xylene isomerization.

U.S. Pat. No. 5,705,726 to Abichandani et al. discloses a xylene isomerization process for mixtures of xylenes and ethylbenzene using a two reactor system wherein the first reactor contains a catalyst effective for ethylbenzene conversion with minimal xylene loss such as selectivated ZSM-5 and the second reactor contains a catalyst effective to catalyze xylene isomerization.

U.S. Pat. No. 4,899,011 to Chu et al. discloses xylene isomerization wherein ethylbenzene and non-aromatics are exhaustively converted using a two component catalyst system wherein the first component can comprise ZSM-5 of greater than 1 micron crystal size and alpha value greater than 100, while the second component can comprise ZSM-5 of less than 1 micron crystal size and alpha value less than 100.

Typical separation procedures for obtaining para-xylene include costly fractional crystallization and adsorptive separation of para-xylene isomers which are customarily recycled. Xylene isomerization units are then required for additional conversion of the recycled xylene isomers in an equilibrium xylene mixture comprising para-xylene. Those skilled in the art appreciate that the expense of the separation process is proportional to the degree of separation required. Therefore, significant cost savings are achieved by increasing selectivity to the para-isomer while maintaining commercially acceptable conversion levels.

Ethylbenzene is an undesirable product formed during xylene synthesis from toluene disproportionation reactions. Its formation strongly affects the purity of para-xylene produced from toluene disproportionation. Minor differences in boiling points between xylenes and ethylbenzene make it difficult to separate the C₈ products by simple distillation. Accordingly, more expensive downstream separation techniques such as crystallization are required to produce high purity para-xylene (greater than 99.5% purity). One approach to minimizing ethylbenzene is disclosed in U.S. Pat. No. 5,498,814 which entails using a single bed platinum-containing toluene disproportionation catalyst for both TDP and ethylbenzene elimination reactions. Incorporation of metal in the TDP catalyst serves to activate conversion of ethylbenzene to benzene and ethane. However, xylene yields can be less than optimal in this single bed process owing to the relative speed with which ethylbenzene is converted to benzene and ethane, compared to conversion of toluene, coupled with the irreversibility of ethylbenzene conversion which “drains” xylene from the overall reaction scheme resulting in undesirable xylene loss.

It is, therefore, highly desirable to provide a process for the production of xylene from toluene at high para-xylene selectivity, while minimizing ethylbenzene formation without reducing xylene yields and maintaining commercially acceptable toluene conversion levels.

### SUMMARY OF THE INVENTION

The present invention relates to the use of a plural catalyst bed process to limit ethylbenzene production from toluene disproportionation processes while maintaining high xylene yields. The first catalyst bed is used to carry out the TDP reaction, while a subsequent catalyst bed for ethylbenzene abatement (located downstream from the first bed) containing selectivated zeolite, selectively eliminates ethylbenzene from effluent of, or effluent derived of, the first bed. The catalyst beds employed in the present invention can be physically located in either two separate reaction vessels or packed together in the same reaction vessel. The catalyst beds can be of different sizes and can operate at different conditions from each other. For purposes of the present invention, it is contemplated that intervening catalyst beds or zones may be placed optionally between the first zone and subsequent zone, i.e., downstream of the first zone and upstream of the subsequent zone.

In one aspect, the present invention relates to a plural stage toluene conversion process for preparing xylenes comprising:

i) contacting in a first stage toluene disproportionation zone a reaction stream comprising toluene and hydrogen with a first stage catalyst comprising catalytic acid molecular sieve which catalyst is substantially free of hydrogenation metal, under toluene disproportionation reaction conditions sufficient to provide a first stage effluent comprising para-xylene and ethylbenzene; and

ii) contacting said effluent from the first stage in a subsequent stage ethylbenzene abatement zone with a subsequent stage catalyst containing porous selectivated catalytic molecular sieve comprising a hydrogenation metal which catalyst is selectivated by treating with a selectivating agent which hinders entry of xylene isomers and permits entry of ethylbenzene into the pores of said subsequent stage catalyst molecular sieve, at reaction conditions sufficient to selectively convert said ethylbenzene to benzene and ethane in the presence of xylenes and toluene to provide a subsequent stage product containing para-xylene and having reduced ethylbenzene content relative to the effluent from the first stage.

The overall single-pass toluene conversion for the process of the present invention can be 5 wt. % or greater, e.g., 5-50 wt. %, preferably 25-50 wt. %, and more preferably 30-40 wt. %. Total xylene yields can be greater than 10 wt. %, preferably greater than 13 wt. %, e.g., 13-15 wt. %. Para-xylene purity (para-xylene/para-xylene+ethylbenzene) can be at least 95%, preferably at least 97%, or even at least 99%. C₈ selectivity in the product from the subsequent stage can range as follows: 70-100 wt. %, preferably 80-95 wt. %, e.g., 85-95 wt. % for para-xylene, 0-15 wt. %, preferably 5-13 wt. %, e.g., 7-13 wt. % for meta-xylene, 1-5 wt.%, preferably 0.5-3 wt. %, e.g., 1-2 wt. % for ortho-xylene, and 0-7 wt. %, preferably 0-3 wt. %, e.g., 0.5-2 wt. % for ethylbenzene, based on total C₈ products.

The first stage and subsequent stage reaction conditions can independently comprise a temperature of 204° to 540° C., preferably 316° to 482° C., a pressure ranging from atmospheric to 1000 psig, preferably from 50 to 400 psig, a WHSV of 0.5 to 100, preferably 3 to 50, and a hydrogen to hydrocarbon molar ratio of 0.5 to 10, preferably 0.5 to 5.

The above and other objects, features and advantages of the present invention will be better understood from the following detailed descriptions, taken in conjunction with the accompanying drawings, all of which are given by illustration only, and are not limitative of the present invention.

### BRIEF DESCRIPTION OF THE DRAWINGS

FIG. 1 depicts a dual reactor process for limiting ethylbenzene production during toluene disproportionation, wherein each reactor contains a single catalyst bed.

FIG. 2 depicts a single reactor process for limiting ethylbenzene production in accordance with the present invention using a single reactor comprising dual catalyst beds.

### DETAILED DESCRIPTION OF THE INVENTION

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

Toluene Disproportionation

Toluene disproportionation is representative of shape selective conversions. Normally a single pass conversion of a toluene stream results in a product stream which includes dimethylbenzenes having alkyl groups at all locations, i.e., ortho-, meta-, and para-xylenes. Furthermore, the xylenes are known to proceed in a reaction which produces unwanted ethylbenzene (EB). In the past, the yield of p-xylene in a single pass has been limited by thermodynamics to approximately 8.2% when isomerization is permitted. This efficiency is significantly reduced by the production of ethylbenzene.

The present invention utilizes a plural bed process to provide higher xylene yields than those obtained at similar p-xylene selectivity and toluene conversion levels with single stage processes. The invention employs at least two beds in order to take advantage of observed toluene disproportionation reactions and ethylbenzene dealkylation reaction kinetics to minimize xylene losses. Kinetics experiments have shown that the following reactions take place during toluene disproportionation:

(1) Toluene→Benzene+Xylenes (slow)

(2) Xylenes⇄Ethylbenzene (equilibrium, fast)

(3) Ethylbenzene→Benzene+Ethane (fastest)

Reaction (1) is the main TDP reaction for xylene formation and is the “slowest” of the three reactions. Reaction (2) involves isomerization of xylenes to ethylbenzene and this reaction is equilibrated within the zeolite pores of the selectivated catalysts. Both reactions (1) and (2) are acid catalyzed and do not require a metal function on the catalyst. If metal is also present, reaction (3) will also take place, a reaction which is “fast” relative to both reactions (1) and (2). Because reaction (3) is irreversible, it serves to “drain” ethylbenzene from the reaction network.

Incorporation of metal in the main TDP catalyst, a single bed process, serves to activate reaction (3) and “drain” ethylbenzene from the reaction network. The loss of ethylbenzene by reaction (3) provides a driving force to further convert xylenes to ethylbenzene via reaction (2). Hence, for a metal-containing single-bed catalyst, xylene losses can be large, e.g., xylene yield losses of 3-4% per pass.

The present invention utilizes a process which separates reactions (1) and (2) from reaction (3). In the first bed, reaction (2) is equilibrium limited and xylene losses by this reaction are minimized. In the second bed, choice of high space velocities (effectively using less catalyst) limits the extent of reactions (1) and (2) so that mainly reaction (3) proceeds to the right. This limits the extent to which xylenes are lost by isomerization reaction (2). By optimizing the first and subsequent catalyst beds and the process conditions, it is contemplated that a range of reactor exit compositions can be obtained, having reduced ethylbenzene content relative to the effluent from the first stage.

Feedstock

The toluene feedstock of the present invention preferably comprises 50% to 100% toluene, more preferably at least 80% toluene. Other compounds such as benzene, xylenes, and trimethylbenzenes may also be present in the toluene feedstock without adversely affecting the present invention.

According to the processes of this invention, the toluene feedstock may also be dried, if desired, in a manner which will minimize moisture entering the reaction zone. Methods known in the art suitable for drying the toluene charge for the present process are numerous. These methods include percolation through any suitable dessicant, for example, silica gel, activated alumina, molecular sieves or other suitable substances, or the use of liquid charge dryers.

Catalysts

First Stage TDP Catalyst

The first stage catalyst molecular sieves used in the present invention are in the hydrogen form and are substantially free of hydrogenation metals. The molecular sieves preferably comprise an intermediate pore-size zeolite having a constraint index within the approximate range of 1 to 12 (e.g., zeolites having less than about 7 angstroms pore size, such as from about 5 to less than 7 angstroms) having a silica to alumina mole ratio of at least about 5, e.g., at least about 12, e.g., at least 20.

The silica to alumina mole ratio referred to may be determined by conventional analysis. This ratio is meant to represent, as closely as possible, the molar ratio in the rigid anionic framework of the zeolite crystal and to exclude silicon and aluminum in the binder or in cationic or other form within the channels.

Examples of intermediate pore size zeolites useful in this invention include ZSM-5 (U.S. Pat. Nos. 3,702,886 and Re. 29,948); ZSM-11 (U.S. Pat. No. 3,709,979); ZSM-5/ZSM-11 intermediate (U.S. Pat. No. 4,229,424); ZSM-12 (U.S. Pat. No. 3,832,449); ZSM-22 (U.S. Pat. No. 4,556,477); ZSM-23 (U.S. Pat. No. 4,076,842); ZSM-35 (U.S. Pat. No. 4,016,245); ZSM-48 (U.S. Pat. No. 4,397,827); ZSM-50 (U.S. Pat. No. 4,640,829); ZSM-57 (U.S. Pat. No. 4,873,067); and/or ZSM-58 (U.S. Pat. No. 4,717,780).

Other zeolites suitable for use in some embodiments of the present invention include zeolite beta (U.S. Pat. Nos. 3,308,069 and Re. 28,341); MCM-22 (U.S. Pat. No. 4,954,325); mordenite, MCM-58 (U.S. Pat. No. 5,569,805); synthetic and natural faujasites, and amorphous or ordered mesoporous materials such as MCM-41 (U.S. Pat. No. 5,098,684).

The alpha value of a catalyst is an approximate indication of the catalytic cracking activity of the catalyst compared to a standard catalyst, and it gives the relative rate constant (rate of normal hexane conversion per volume of catalyst per unit time). It is based on the activity of the amorphous silica-alumina cracking catalyst taken as an alpha of 1 (Rate Constant=0.016 sec⁻¹). The alpha test is described in U.S. Pat. No. 3,354,078 and in the Journal of Catalysis, 4, 522-529 (1965); 6, 278 (1966); and 61, 395 (1980), each incorporated herein by reference as to that description. It is noted that intrinsic rate constants for many acid-catalyzed reactions are proportional to the alpha value for a particular crystalline silicate catalyst (see “The Active Site of Acidic Aluminosilicate Catalysts,” Nature, Vol. 309, No. 5959, 589-591, (1984)). The experimental conditions of the test used herein include a constant temperature of 538° C. and a variable flow rate as described in detail in the Journal of Catalysis, 61, 395 (1980). The catalysts employed in the process of the present invention can have an alpha value greater than 1, preferably greater than 100, for example, about 150-2000, and a silica-alumina ratio less than 100, preferably 20-80. The alpha value of the catalyst may be increased by initially treating the catalyst with nitric acid or by mild steaming before selectivation as discussed in U.S. Pat. No. 4,326,994.

The crystal size of zeolites used herein is preferably greater than 0.1 micron. The accurate measurement of crystal size of zeolite materials is frequently very difficult. Microscopy methods such as SEM and TEM are often used, but these methods require measurements on a large number of crystals and for each crystal measured, values may be required in up to three dimensions. For ZSM-5 materials described in the examples below, estimates were made of the effective average crystal size by measuring the rate of sorption of 2,2-dimethylbutane at 90° C. and 60 torr hydrocarbon pressure. The crystal size is computed by applying the diffusion equation given by J. Crank, The Mathematics of Diffusion, Oxford at the Clarendon Press, 52-56 (1957), for the rate of sorbate uptake by a solid whose diffusion properties can be approximated by a plane sheet model. In addition, the diffusion constant of 2,2-dimethylbutane, D, under these conditions is taken to be 1.5×10⁻¹⁴ cm²/sec. The relation between crystal size measured in microns, d, and diffusion time measured in minutes, t0.3, the time required for uptake of 30% of capacity of hydrocarbon, is: d=0.0704×t0.3^{1/2}.

In the present case these measurements have been made on a computer controlled, thermogravimetric electrobalance, but there are numerous ways one skilled in the art could obtain the data. For example, larger crystal material used herein can have sorption time, t0.3 of 497 minutes which gives a calculated crystal size of 1.6 microns. Smaller crystal material can have a sorption time of 7.8 minutes, and a calculated crystal size of 0.20 micron.

The catalysts of the present invention can optionally be employed in combination with a support or binder material. The binder is preferably an inert, non-alumina containing material, such as a porous inorganic oxide support or a clay binder.

One such preferred inorganic oxide is silica. Other examples of such binder material include, but are not limited to zirconia, magnesia, titania, thoria and boria. These materials can be utilized in the form of a dried inorganic oxide gel or as a gelatinous precipitate. Suitable examples of clay binder materials include, but are not limited to, bentonite and kieselguhr. The relative proportion of catalyst to binder material to be utilized is from about 30 wt. % to about 98 wt. %. A proportion of catalyst to binder from about 50 wt. % to about 80 wt. % is more preferred. The bound catalyst can be in the form of an extrudate, beads or fluidizable microspheres.

An especially preferred zeolite suitable for use in the process of the present invention is a synthetic porous crystalline material having the structure of ZSM-5 and a composition involving the molar relationship:

X₂O₃:(n)YO₂,

wherein X is a trivalent element; Y is a tetravalent element; and n is greater than about 12, and wherein the crystals have a major dimension of at least about 0.5 micron, preferably at least about 1 micron, and a surface YO₂/X₂O₃ ratio which is no more than 20%, preferably no more than 10%, greater than the bulk YO₂/X₂O₃ ratio of the crystal. Such large crystal ZSM-5 can have SiO₂/Al₂O₃ ratios of 100 or lower, preferably 40 or lower, say, 25-40, and is prepared by using amino acids as the directing agent. The resultant large ZSM-5 crystals have essentially the same SiO₂/Al₂O₃ ratios in bulk (as measured by elemental and NMR analyses) and on the surface (as measured by x-ray photoelectron spectroscopy analysis (XPS) which determines the surface concentration and oxidation state of all detectable elements.) The crystalline material preferably exhibits a sorption time for 2,2-dimethylbutane for 30% of its capacity, as measured at 120° C. and 60 torr (8 kPa) hydrocarbon pressure, of at least 5 minutes, more preferably at least 10 minutes.

The large crystal porous crystalline material having the structure of ZSM-5 can be prepared by a process comprising the steps of forming a reaction mixture containing sources of an alkali or alkaline-earth metal oxide, a trivalent metal oxide X₂O₃, a tetravalent metal oxide YO₂, water, and an amino acid or salt thereof having the formula:

R₁—(CH₂)_{Z}—CR₂HCOOA

wherein R₁ is NH₂, NHR₃ where R₃ is an adamantyl or cyclic alkyl group, or a carboxylic acid group or salt thereof; R₂ is H, alkyl, aryl, alkaryl, NH₂ or NHR₃ where R₃ is an adamantyl or cyclic alkyl group; provided that at least one of R₁ and R₂ is NH₂ or NHR₃, A is H or a metal and z is from 0 to 15, provided that at least one of R₁ and R₂ is NH₂ or NHR₃, and crystallizing the reaction mixture to produce said porous crystalline material. Preferably, the amino acid or salt thereof is selected from 6-aminohexanoic acid, lysine, glutamic acid and monosodium glutamate.

Further description of these large crystal ZSM-5 materials can be found in PCT Application 96/40426, published Dec. 19, 1996.

Subsequent Stage Ethylbenzene Abatement Catalyst

The catalyst of the subsequent stage can have the same characteristics within the range of parameters given for the first stage catalyst described above, except that the ethylbenzene abatement catalyst is associated with a hydrogenation component (hydrogenation-dehydrogenation component). Examples of such components include the oxide, hydroxide, sulfide, or free metal (i.e., zero valent) forms of Group VIIIA metals (i.e., Pt, Pd, Ir, Rh, Os, Ru, Ni, Co and Fe), Group VIIA metals (i.e., Mn, Tc, and Re), Group VIA metals (i.e., Cr, Mo, and W), Group VB metals (i.e., Sb and Bi), Group IVB metals (i.e., Sn and Pb), Group IIB metals (i.e., Ga and In), and Group IB metals (i.e., Cu, Ag and Au). Noble metals (i.e., Pt, Pd, Ir, Rh, Os and Ru) are preferred hydrogenation components. Combinations of catalytic forms of such noble or non-noble metal, such as combinations of Pt with Sn, may be used. The metal may be in a reduced valence state, e.g., when this component is in the form of an oxide or hydroxide. The reduced valence state of this metal may be attained, in situ, during the course of a reaction, when a reducing agent, such as hydrogen, is included in the feed to the reaction.

The hydrogenation component may be incorporated into the catalyst by methods known in the art, such as ion exchange, impregnation or physical admixture. For example, solutions of appropriate metal salts may be contacted with the remaining catalyst components, either before or after selectivation of the catalyst, under conditions sufficient to combine the respective components. The metal containing salt may be water soluble. Examples of such salts include chloroplatinic acid, tetraamineplatinum complexes, platinum chloride, tin sulfate and tin chloride. The metal may be incorporated in the form of a cationic, anionic or neutral complex such as Pt(NH₃)₂²⁺ and cationic complexes of this type will be found convenient for exchanging metals onto the zeolite. For example, a platinum modified catalyst can be prepared by first adding the catalyst to a solution of ammonium nitrate in order to convert the catalyst to the ammonium form. The catalyst is subsequently contacted with an aqueous solution of tetraamine platinum (II) nitrate or tetraaamine platinum (II) chloride. Anionic complexes such as the vanadate or metatungstate ions are also useful for impregnating metals into the zeolites. Incorporation may be undertaken in accordance with the invention of U.S. Pat. No. 4,312,790. After incorporation of the metal, the catalyst can then be filtered, washed with water and calcined at temperatures of from about 250° C. to about 500° C.

The amount of hydrogenation component may be that amount which imparts or increases the catalytic ability of the overall catalyst to catalytically hydrogenate or dehydrogenate an organic compound under sufficient hydrogenation or dehydrogenation conditions, e.g., hydrogenate ethylene to ethane. This amount is referred to as a catalytic amount. The amount of the hydrogenation component may be from 0.001 to 10 percent by weight, although this will, of course, vary with the nature of the component, with less of the highly active noble metals, particularly platinum, being required than of the less active base metals.

The preferred binder or support for the ethylbenzene abatement catalyst of the subsequent stage is silica. Without intending to be bound thereby, it is believed that alumina binder catalyzed xylene isomerization reactions are further reduced through the use of inert silica binding for the ethylbenzene abatement catalyst. Thus, the binder for said catalyst may contain no intentionally added alumina.

Catalyst Selectivation

First Stage Toluene Disproportionation Catalyst

In one aspect of the present invention, the first stage acidic catalyst can be a selectivated catalyst treated in situ or ex situ with controlled coking or preferably, a high-efficiency para-xylene selectivating agent.

Selectivation by coking can be employed as a means to selectivate the first stage catalyst, e.g., by in situ trim-selectivation by coke trim-selectivation wherein an organic compound is decomposed in the presence of the modified catalytic molecular sieve, at conditions suitable for decomposing the organic compound. Alternatively, the trim-selectivation can be performed by exposing the catalytic molecular sieve to a reaction stream that includes an alkylaromatic and a trim-selectivating agent selected from a group of compounds including a large variety of silicon-containing compounds, at reaction conditions.

Use of a high efficiency para-xylene selectivating agent advantageously increases the para-selectivity of the catalyst, and therefore, the efficiency of para-xylene production during the conversion of toluene to xylene. As used herein, the term “high efficiency, p-xylene selectivating agent” is used to indicate substances which will increase the para-selectivity of a catalytic molecular sieve to the stated levels while maintaining commercially acceptable toluene to xylene conversion levels. Such substances suited to in situ treatment include volatile organosilicon compounds with sufficient vapor pressure for proper deposition under conversion conditions, i.e., temperatures ranging from 100° C. to 600° C., preferably from 300° C. to 500° C.; pressures ranging from 0 to 2000 psig, preferably from 15 to 800 psig; a mole ratio of hydrogen to hydrocarbons from greater than 0 to 10, preferably from 1 to 4; at a weight hourly space velocity (WHSV) from 0.1 to 100 hr⁻¹, preferably from 1 to 20 hr⁻¹ Upon thermolysis, a siliceous coating is deposited on the zeolite surface, eliminating surface activity and enhancing shape-selectivity.

The volatile organosilicon compounds comprise polysiloxanes including silicones, and also siloxanes, and silanes including disilanes and alkoxysilanes. Silicone compounds which can be used in the present invention can be characterized by the general formula: —R₁R₂SiO— where R₁ is hydrogen, fluorine, hydroxy, alkyl, aralkyl, alkaryl or fluoro-alkyl. The hydrocarbon substituents generally contain from 1 to 10 carbon atoms and preferably are methyl or ethyl groups. R₂ is selected from the same group as R₁, and n is an integer of at least 2 and generally in the range of 2 to 1000. The molecular weight of the silicone compound employed is generally between 80 and 20,000 and preferably with the range of 150 to 10,000. Representative silicone compounds include dimethylsilicone, diethylsilicone, phenylmethylsilicone, methylhydrogensilicone, ethylhydrogensilicone, phenylhydrogensilicone, methylethylsilicone, phenylethylsilicone, diphenylsilicone, methyltrifluoropropylsilicone, ethyltrifluoropropylsilicone, tetrachlorophenylmethylsilicone, tetrachlorophenylethylsilicone, tetrachlorophenylhydrogen silicone, tetrachlorophenylphenyl silicone, methylvinylsilicone and ethylvinylsilicone. The silicone compound need not be linear but may be cyclic as, for example, hexamethylcyclotrisiloxane, hexaethylcyclotrisiloxane, octamethylcyclotetrasiloxane, octaethylcyclotetrasiloxane, hexaphenylcyclotrisiloxane, hexaphenylcyclotrisiloxane, octaphenylcylotetrasiloxane, decamethylcyclopentasiloxane, hexamethyldisiloxane, octamethyltrisiloxane, and decamethyltetrasiloxane. Mixtures of these compounds may also be used as well as silicones with other functional groups.

Useful silane, disilanes or alkoxysilanes include organic substituted silanes having the general formula: —RR₁R₂R₃Si— wherein R is a reactive group such as hydrogen, alkoxy, halogen, carboxy, amino, or acetamide trialkylsilyl. R₁, R₂ and R₃ can be the same as R or an organic radical which may include alkyl of from 1 up to 40 carbon atoms, alkyl or aryl carboxylic acid wherein the organic portion of the alkyl contains 1 to 30 carbon atoms and the aryl group contains 6 to 24 carbon atoms, aryl groups of 6 to 24 carbons which may be further substituted, alkylaryl and arylalkyl groups containing 7 to 30 carbon atoms. Preferably, the alkyl group of an alkyl silane is between 1 and 4 carbon atoms in chain length. Mixtures may also be used.

The silanes or disilanes include, as non-limiting examples, dimethylphenylsilane, phenyltrimethylsilane, triethylsilane and hexamethyldisilane. Useful alkoxysilanes are those with at least one silicon-hydrogen bond.

Preferred silicon-containing selectivating agents include dimethylphenylmethylpolysiloxane (e.g., Dow-550™) and phenylmethyl polysiloxane (e.g., Dow-710™). Dow-550™ and Dow-710™ are available from Dow Chemical Company, Midland, Mich.

While not wishing to be bound by theory, it is believed that the advantages of selectivating the first stage TDP catalyst are obtained by rendering acid sites on the external surfaces of the catalyst substantially inaccessible to reactants while increasing catalyst tortuousity. Acid sites existing on the external surface of the catalyst are believed to isomerize the para-xylene exiting the catalyst pores back to an equilibrium level with the other two isomers thereby reducing the amount of para-xylene in the xylenes to only about 24%. By reducing the availability of these acid sites to the para-xylene exiting the pores of the catalyst, the relatively high level of para-xylene can be maintained. It is believed that the high efficiency, p-xylene selectivity agents block or otherwise render these external acid sites unavailable to the para-xylene by chemically modifying the sites.

Preferably, the kinetic diameter of the high efficiency, p-xylene selectivating agent is larger than the zeolite pore diameter, in order to avoid reducing the internal activity of the catalyst.

It is believed that the presence of hydrogen in the reaction zone is helpful to maintain the desired high yields of para-xylene when a silicone compound is used as the high-efficiency para-xylene selectivating agent. The importance of the hydrogen in the feedstock may be reduced in alternative embodiments by using a high efficiency para-xylene selectivating agent comprising silane or some other compound which effectively renders the isomerizing acid sites on the external surface of the catalyst inaccessible.

One way of selectivating the catalyst utilizes a high efficiency para-xylene selectivating agent comprising a silicone compound wherein the silicone compound is introduced by co-feeding, for example, at least one silicone compound with the toluene feedstock over a conversion catalyst at reaction conditions. In addition to such in situ treatments during TDP, the selectivation can be carried out on the catalyst prior to toluene disproportionation, e.g., by conventional ex situ treatments of the catalyst before loading into the reactor, or in situ treatment of the loaded catalyst prior to contacting the catalyst with toluene feedstock. Multiple ex situ treatments, say 2 to 6 treatments, preferably 2 to 4 treatments, have been found especially useful to selectivate the catalyst.

Subsequent Stage Ethylbenzene Abatement Catalyst

Included in the present invention is a process for the selective disproportionation of a toluene feedstock into para-xylene utilizing a separate, selectivated, high-efficiency ethylbenzene abatement selectivating agent in the subsequent stage. The selectivating agent advantageously increases the ethylbenzene abatement activity of the subsequent stage catalyst, and therefore, the efficiency of the overall process inasmuch as the need to separate out ethylbenzene from the process product is reduced or eliminated. As used herein, the term “high-efficiency, ethylbenzene abatement selectivating agent” is used to indicate substances which will hinder entry of xylene isomers in the catalyst pore system, while permitting entry of ethylbenzene for hydrogenation to benzene and ethane, resulting in reduction or elimination of ethylbenzene while minimizing xylene yield loss. Such substances include volatile organosilicon compounds such as those noted above which are used to treat the first stage catalyst. In addition, selectivation by coking as described above with respect to the first stage catalyst can also be employed as a means to selectivate the subsequent stage catalyst, e.g., by coke trim-selectivation wherein an organic compound is decomposed in the presence of the modified catalytic molecular sieve, at conditions suitable for decomposing the organic compound.

Organic materials, thermally decomposable to provide coke trimming, encompass a wide variety of compounds including, by way of example, hydrocarbons, such as paraffinic, cycloparaffmic, olefinic, cycloolefinic and aromatic; oxygen-containing organic compounds such as alcohols, aldehydes, ethers, ketones and phenols; heterocyclics such as furans, thiophenes, pyrroles and pyridines. Usually, it is contemplated that a thermally decomposable hydrocarbon, such as an alkyl-substituted aromatic, will be the source of coke, most preferably the alkylaromatic being subjected to conversion itself. In the latter case, the alkylaromatic is initially brought into contact with the catalyst under conditions of temperature and hydrogen concentration amenable to rapid coke formation. Typically, coke trimming is conducted at conditions outside the operating parameters used during the main time span of the catalytic cycle. When the desired coke deposition has been effected, the alkylaromatic feed is continued in contact with the coke-containing catalyst under conditions of temperature and hydrogen concentration conducive to disproportionation, with a greatly reduced coking rate. Alternatively, the trim-selectivation can be performed by exposing the catalytic molecular sieve to a reaction stream that includes an alkylaromatic and a trim-selectivating agent selected from a group of compounds including a large variety of silicon-containing compounds, at reaction conditions.

Relative Catalyst Bed Sizes

The subsequent stage catalyst bed is generally much smaller than the first catalyst bed because ethylbenzene abatement reactions are considerably faster than toluene disproportionation and/or xylene isomerization reactions. In general, the weight ratio of first catalyst bed to subsequent catalyst bed can range between >1 to 10, preferably 2 to 5.

Process Conditions

First Stage

Operating conditions employed in the process of the present invention will affect the para-selectivity and toluene conversion in the first stage. The first stage reactor is operated at conditions which maximize xylene yield and para-xylene selectivity. Such conditions include the temperature, pressure, space velocity, molar ratio of the reactants, and the hydrogen to hydrocarbon mole ratio, H₂/HC. It has also been observed that an increased space velocity (WHSV) can enhance the para-selectivity of the modified catalyst in toluene disproportionation reactions. This characteristic of the modified catalyst allows for substantially improved throughput when compared to current commercial practices. In addition, it has been observed that the disproportionation process can be performed using H₂ as a diluent, thereby dramatically increasing the cycle length of the catalyst.

First stage reaction conditions effective for accomplishing high para-selectivity and acceptable toluene disproportionation conversion levels can include reactor inlet temperatures ranging from 90° to 1110° F., preferably 6600 to 1000° F., a pressure ranging from atmospheric to 5000 psia, preferably from 1000 to 5000 psia, a WHSV of 0.1 to 20, preferably from 2 to 10, and a hydrogen to hydrocarbon molar ratio of 0.1-20, preferably from 0.5 to 10.

Subsequent Stage

Operating conditions employed in the subsequent stage wherein ethylbenzene abatement occurs can be independent of those conditions given above for the first stage. Generally, ethylbenzene conversion conditions are constrained by the need to hold conversion of xylenes to other compounds to acceptable levels. Thus, operating conditions are selected to balance the disadvantages of xylene loss by transalkylation with the conversion of ethylbenzene.

Subsequent stage operating conditions of the present invention can include a temperature of 400° to 1000° F., preferably 600° to 900° F., a pressure ranging from 15 to 1015 psia, preferably 65 to 415 psia, a WHSV of between 0.1 to 200 hr⁻¹, preferably between 3 and 50 hr⁻¹, and a hydrogen to hydrocarbon molar ratio of between 0.1 and 10, preferably between 0.5 and 5.

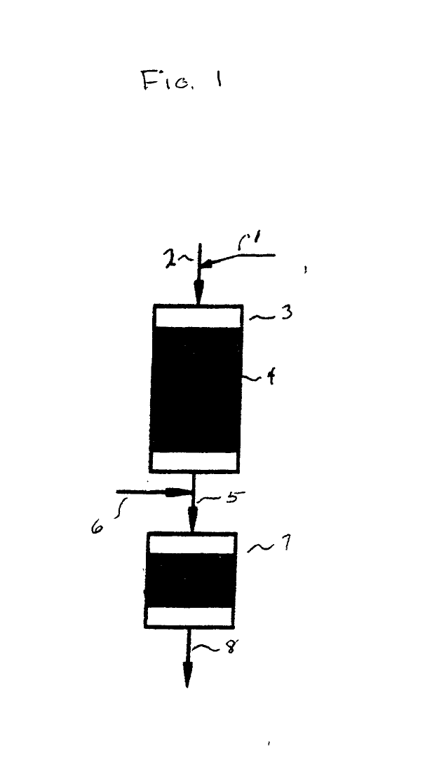

In FIG. 1, depicting a dual-reactor scenario for limiting ethylbenzene production from TDP reactors, a toluene-containing feed 1 which may contain hydrogen is directed via line 2 to first stage TDP reactor 3 where it contacts TDP catalyst 4 and is converted to a mixture containing toluene, xylenes, and ethylbenzene which exits the reactor as first stage effluent via line 5. Additional hydrogen may be added to the first stage effluent through line 6 and the first stage effluent is thence directed to the subsequent stage reactor 7 where it contacts a catalyst bed which is maintained at ethylbenzene conversion conditions which converts ethylbenzene to ethane and benzene while minimizing xylene conversion and preferably converting toluene to xylenes. The product of the subsequent stage which contains lesser amounts of ethylbenzene than the first stage effluent exits through line 8.

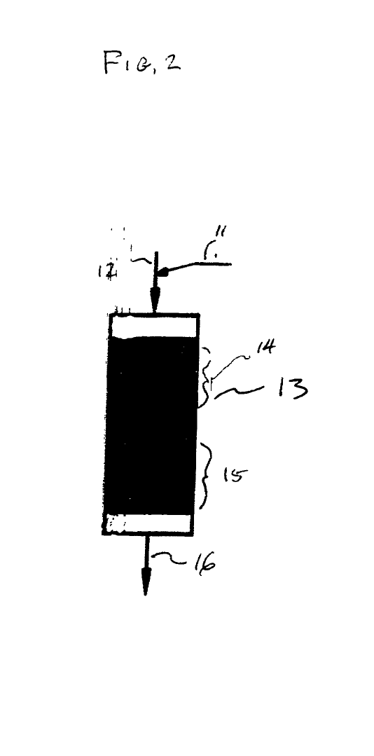

In FIG. 2 which depicts dual catalyst beds in a single reactor scenario to limit ethylbenzene production from TDP reactors, a toluene-containing feed 11 which may contain hydrogen is directed via line 12 to the first stage of combined TDP/ethylbenzene abatement reactor 13 where it contacts TDP catalyst 14 and is converted to a mixture containing toluene, xylenes, and ethylbenzene which then enters a subsequent stage ethylbenzene abatement catalyst bed 15 which is maintained at ethylbenzene conversion conditions to convert ethylbenzene to ethane and benzene while minimizing xylene conversion and preferably converting toluene to xylenes. The product of the subsequent stage which contains lesser amounts of ethylbenzene than the first stage effluent exits through line 16.

All of the foregoing U.S. patents are incorporated herein by reference.

The following examples will serve to further illustrate processes and some advantages of the present invention.

### Example 1

Preparation of ZSM-5

A 5 gallon preparation of ZSM-5 was prepared as follows: 1.0 part of Al₂SO₄×H₂O was dissolved in 8.0 parts of H₂O. To this solution were added 1.98 parts of 50% sodium hydroxide solution. A solution obtained by dissolving 1.12 parts of the monosodium salt of glutamic acid in 2.71 parts of H₂O was added to the above solution. To this mixture were added 4.03 parts of Ultrasil™, precipitated silica available from J. M. Huber Corp. of Macon, Ga. The slurry was thoroughly mixed and then 8.0 grams of ZSM-5 seeds (solids basis) slurried in 2.28 parts of H₂O were added to the mixture and the final slurry mixed for 30 minutes. The composition of the reaction mixture in mole ratios was as follows:

SiO₂/Al₂O₃=36.0

OH⁻/SiO₂=0.24

Na/SiO₂=0.50

R/SiO₂=0.10

H₂O=12.8

The mixture was crystallized in a stainless steel reactor, with stirring, at 100 rpm, at 156° C. for 60 hours.

The X-ray analysis showed a crystalline ZSM-5 material.

The composition of the material in wt. % was:

N=0.04

Na=2.44

Al₂O₃=4.82

SiO₂=83.0

SiO₂/Al₂O₃=29.6

### Example 2

Preparation of Hydrogen Form of ZSM-5 (HZSM-5)

25 grams of the product of Example 1 were calcined for 16 hours at 538° C. The calcined material was contacted with a 10% NH₄Cl solution at 85° C. for 1 hour, three times with constant stirring. The sodium level was 0.02%. The material was then calcined for three hours at 538° C. to convert it to the hydrogen form. The alpha value of this material was determined to be 1454.

### Example 3

Preparation of Silica Extrudate of Hydrogen Form of ZSM-5 (HZSM-5)

The ZSM-5 drycake of Example 3 was mixed with Ultrasil™ VN3SP silica and Ludox HS-40™ (DuPont) in proportion to give 65% ZSM-5/17.5% SiO₂ ex Ultrasil/17.5% SiO₂ ex Ludox™ on 100% solids basis. Deionized water and 3% NaOH (on 100% solids basis) were added to give an extrudable mull and the mix was extruded to 1/16 inch diameter. The extrudate was dried at 120° C. and charged to a column. It was exchanged two times for one hour at room temperature with 1N NH₄NO₃ solution (5 grams solution per gram of extrudate), washed with deionized water, and dried at 120° C. The extrudate was then charged to a muffle furnace and calcined in flowing nitrogen at 540° C. for three hours. It was again charged to a column and exchanged two times with 1N NH₄NO₃ solution (5 grams solution per gram of extrudate), washed with deionized water, and dried at 120° C. It was then charged to a muffle furnace and calcined in flowing air at 540° C. for three hours. The properties of the catalyst were analyzed as follows:

Particle density, g/cc 2.32

Real density, g/cc 1.14

Surface area, m²/g 205

Alpha 1017

Na, ppm 70

### Example 4

Ex Situ Selectivation of Silica Extrudate of Hydrogen Form of ZSM-5 (HZSM-5)—Bed 1 Catalyst

The extrudates from Example 3 were ex-situ selectivated using Dow 550™. The procedure used for catalyst selectivation was a multiple silicone coating procedure (pore filling technique) with Dow 550™/n-decane. 30 grams of extrudates were impregnated in 1.0:1.1 n-decane:Dow ₅₅₀™ weight ratio at room temperature. After impregnation, the solvent was stripped in a Bucci Rotovap™ unit and calcined in a tube furnace with nitrogen (25 to 125° C. at 1° C./minute, held for two hours; 130 to 540° C. at 2° C./minute, held for two hours; cooled down to 300° C.) followed by air calcination (300 to 540° C. at 2° C./minute, held for six hours). The same procedure was followed for the remaining selectivation cycles (2nd, 3rd, 4th, etc. cycles abbreviated as 1X, 2X, 3X, 4X, etc.). After each selectivation cycle, a small sample was removed for catalytic evaluations.

### Example 5

Preparation of Pt-ZSM-5 Silica Extrudate—Bed 2 Catalyst

Platinum was introduced in the silica extrudate of Example 3 by ion-exchange using a Pt(NH₃)₄(NO₃)₂ solution. 5.5 grams of the extrudate were buffered in 25 ml, 1M NH₄NO₃ solution. The platinum salt solution was added dropwise to the buffered catalyst solution with constant stirring. The ion-exchanged catalyst was then filtered and dried at room temperature; and calcined in an electric furnace with air (calcined at 25 to 150° C. at 5° C./minute, held for 1 hour; calcined again at 150 to 350° C. at 1° C./minute, held for 5 hours; and cooled to room temperature). The nominal platinum loading of this catalyst was 0.1 weight percent Pt.

### Example 6

Ex Situ Selectivation of Silica Extrudate of Platinum Form of ZSM-5 (PtZSM-5)—Bed 2 Catalyst

The platinum-containing ZSM-5 catalyst of Example 5 was selectivated using multiple silicone treatments as outlined in Example 4 above. The multiple-selectivated, platinum-containing catalyst was used as the subsequent bed ethylbenzene abatement catalyst (Bed 2).

### Example 7

Catalytic Testing of Selectivated Catalyst: Bed 1 Catalyst Only

The selectivated catalyst of Example 4 was tested for toluene disproportionation activity (Bed 1) in an automated unit with on-line gas chromatography (GC) sampling. In this test, 2.0 grams of the selectivated extrudate were mixed with inert sand and loaded into a 0.375″ diameter stainless steel tube reactor, dried in hydrogen to reaction temperature and the toluene/hydrogen feed was then introduced at 3 WHSV, 280 psig and 2 H₂/HC, with temperature varied to achieve ca. 30% toluene conversion. The reactor effluent form the first bed catalyst was sampled and analyzed using on-line GC.

A summary of the catalytic data for this catalyst bed is presented in Table 1 below. This product distribution is customary of TDP reactions over selectivated HZSM-5 catalysts. In particular, it was observed that ethylbenzene accounts for 3% of the total C₈ products (p-, m-, o-xylene and ethylbenzene) formed during the reaction.

TABLE 1

| | Bed 1 Effluent/H-ZSM-5 (3X) |
|---|---|
| | Example 7 |
| Temperature, ° C. | 407 |
| Catalyst Weight, grams | 2.0 |
| WHSV, hr⁻¹ | 3 |
| H₂:HC (feed) | 2 |
| Pressure, psig | 280 |
| Toluene Conversion, weight percent | 28.0 |
| p-Xylene Selectivity, weight percent | 93.0 |
| Xylene Yield, weight percent | 12.2 |
| Benzene/Xylene (molar) | 1.8 |
| C₅⁺ | 1.9 |
| C₉⁺ | 0.4 |
| Selectivity of C₈ Products: | |
| Ethylbenzene selectivity, wt. % | 3.0 |
| p-xylene selectivity, wt. % | 90.0 |
| m-xylene selectivity, wt. % | 6.3 |
| o-xylene selectivity, wt. % | 0.7 |

### Example 8

Catalytic Testing of Selectivated Catalyst: Bed 1 & Bed 2 Cascade Evaluation (Same Conditions in Both Catalyst Beds)

0.5 gram of the platinum containing, 2X-selectivated catalyst from Example 6 was mixed with inert sand and loaded into a 0.375″ diameter stainless steel tube second reactor and dried in hydrogen to reaction temperature. Next, the effluent from the first reactor was diverted to the inlet of the second reactor. Since there is less catalyst in bed 2, the effective space velocity over this catalyst was about 12 hr⁻¹. In this example, catalyst bed 2 temperature was matched to that of catalyst bed 1 in order to simulate combined bed operation as depicted in FIG. 2. The reactor effluent from the second bed catalyst was sampled and analyzed using on-line GC. Performance data for the dual catalyst bed is presented in Table 2, below. Comparison of Tables 1 and 2 immediately illustrates that the second catalyst bed is highly effective for eliminating ethylbenzene from the product pool. As expected, reduction in ethylbenzene levels is accompanied by a corresponding increase in C₅⁺ product from the cracking/hydrogenation reactions. It is further observed that additional TDP reactions also take place in the second bed and increase the overall toluene conversion from 28% to 34.5%. One of the most significant effects of this reactor/catalyst arrangement is that one is able to maintain high (13-15 wt. %) xylene yields, while eliminating ethylbenzene from the reaction products. The small drop in p-xylene selectivity is due to the lower selectivity of the platinum-containing catalyst used here, which only has two ex-situ selectivation treatments. p-Xylene selectivity loss can be minimized by using a more highly selectivated platinum/ZSM-5 catalyst, such as a 3-4X catalyst.

### Example 9

Catalytic Testing of Selectivated Catalysts: Bed 1 & Bed 2 Cascade Evaluation (Different Conditions in Each Catalyst Bed)

The temperature of the second catalyst bed was lowered, while continuing to maintain catalyst in bed 1 at conditions discussed in Example 7. This provides an illustrative example of the dual-reactor concept presented in FIG. 1, in which two separate reactors are used to permit each bed to operate at different conditions.

Performance data for the lower temperature (393° C.) example are also presented in Table 2. Lowering the temperature of bed 2 reduced the overall toluene conversion because the activity of the TDP reactions is reduced. Ethylbenzene cracking reactions remained active and continued to significantly reduce ethylbenzene in the reactor effluent. The xylene yields and p-xylene selectivity remained high.

TABLE 2

| | Bed 2 Effluent | Bed 2 Effluent |
|---|---|---|
| | Pt/H-ZSM-5 (3X) | Pt/H-ZSM-5 (2X) |
| | Example 8 | Example 9 |
| Temperature, ° C. | 407 | 393 |
| Catalyst Weight, grams | 0.5 | 0.5 |
| WHSV, hr⁻¹ | 12 | 12 |
| H₂:HC (feed) | 2 | 2 |
| Pressure, psig | 280 | 280 |
| Toluene Conversion, wt. % | 34.5 | 32.5 |
| p-Xylene Selectivity, wt. % | 86.0 | 89.1 |
| Xylene Yield, wt. % | 13.8 | 13.1 |
| Benzene/Xylene (molar) | 1.7 | 1.7 |
| C₅⁺ | 5.0 | 4.4 |
| C₉⁺ | 0.4 | 0.5 |
| Selectivity of C₈ Products: | | |
| Ethylbenzene | 0.3 | 0.7 |
| selectivity, wt. % | | |
| p-xylene selectivity, wt. % | 85.7 | 88.3 |
| m-xylene selectivity, wt. % | 12.3 | 9.7 |
| o-xylene selectivity, wt. % | 1.7 | 1.3 |

It is claimed:

1. A plural stage toluene conversion process for preparing xylenes comprising:
i) contacting in a first stage toluene disproportionation zone a reaction stream comprising toluene and hydrogen with a first stage catalyst comprising catalytic acid molecular sieve which catalyst is substantially free of hydrogenation metal, under toluene disproportionation reaction conditions sufficient to provide a first stage effluent comprising para-xylene and ethylbenzene; and
ii) contacting said effluent from the first stage in a subsequent stage ethylbenzene abatement zone with a subsequent stage catalyst containing porous selectivated catalytic molecular sieve comprising a hydrogenation metal which catalyst is selectivated by treating with a selectivating agent which hinders entry of xylene isomers and permits entry of ethylbenzene into the pores of said subsequent stage catalyst molecular sieve, at reaction conditions sufficient to selectively convert said ethylbenzene to benzene and ethane in the presence of xylenes and toluene to provide a subsequent stage product containing para-xylene and having reduced ethylbenzene content relative to the effluent from said first stage.

2. The process of claim 1 wherein
a) said first stage catalytic acid molecular sieve is a selectivated shape-selective zeolite having a constraint index of 1 to 12;
b) said subsequent stage catalytic acid molecular sieve is a shape-selective zeolite having a constraint index of 1 to 12, and said hydrogenation metal is selected from the group selected from Group VIIIA, Group VIIA, Group VIA, Group VB, Group IVB, Group IIIB, Group IB, and combinations thereof of the Periodic Table, and
c) said first stage and said subsequent stage reaction conditions independently comprise a temperature of 204° to 540° C., a pressure ranging from atmospheric to 1000 psig, a WHSV of 0.5 to 100, and a hydrogen to hydrocarbon molar ratio of 0.5-10, said first stage and said subsequent stage process conditions further providing an overall single-pass toluene conversion of at least 5 wt. %, total xylene yields greater than 10 wt. %, para-xylene purity (para-xylene/para-xylene+ethylbenzene) of at least 95%, and/or C₈ selectivity in the product from the subsequent stage ranging from 70-100 wt. % for para-xylene and 0-7 wt. % ethylbenzene, based on total C₈ products.

3. The process of claim 2 wherein said first stage and said subsequent stage reaction conditions independently comprise a temperature of 316° to 482° C., a pressure ranging from 50 to 400 psig, a WHSV of 3 to 50, and a hydrogen to hydrocarbon molar ratio of 0.5 to 5, said first stage and said subsequent stage process conditions further providing an overall single-pass toluene conversion of 25-50 wt. %, total xylene yields greater than 13 wt. %, para-xylene purity (para-xylene/para-xylene+ethylbenzene) of at least 97%, and/or C₈ selectivity in the product from the subsequent stage ranging from 80-95 wt. % for para-xylene and 0-3 wt. % ethylbenzene, based on total C₈ products.

4. The process of claim 1 wherein said first stage catalyst comprises a zeolite selected from the group consisting of ZSM-5, ZSM-11, ZSM-5/ZSM-11 intermediate, ZSM-12, ZSM-22, ZSM-23, ZSM-35, ZSM-48, ZSM-50, ZSM-57 and ZSM-58, and having a crystal size of at least 0.5 microns, and said subsequent stage catalyst comprises a zeolite selected from the group consisting of ZSM-5, ZSM-11, ZSM-5/ZSM-11 intermediate, ZSM-12, ZSM-22, ZSM-23, ZSM-35, ZSM-48, ZSM-50, ZSM-57 and ZSM-58, having a crystal size of at least 0.5 micron, and a hydrogenation component selected from the group consisting of platinum and palladium.

5. The process of claim 2 wherein at least one of said first stage catalyst and said subsequent stage catalyst comprises ZSM-5 zeolite crystals having a major dimension of at least about 1 micron, a surface SiO₂/Al₂O₃ ratio which is no more than 20% greater than the bulk SiO₂/Al₂O₃ ratio of the crystal, and a SiO₂/Al₂O₃ ratio of 25-40.

6. The process of claim 2 wherein said first stage catalyst is selectivated in situ with a mixture containing a high-efficiency, para-xylene selectivating agent selected from the group consisting of polysiloxanes, siloxanes, silanes, disilanes and alkoxysilanes.

7. The process of claim 2 wherein said first stage catalyst is selectivated ex situ with a mixture containing a high-efficiency, para-xylene selectivating agent selected from the group consisting of polysiloxanes, siloxanes, silanes, disilanes and alkoxysilanes.

8. The process of claim 2 wherein said first stage catalyst is selectivated by a process selected from the group consisting of in situ coke deposition and ex situ coke deposition.

9. The process of claim 1 wherein said subsequent stage catalyst is selectivated in situ by treating with a selectivating agent selected from the group consisting of polysiloxanes, siloxanes, silanes, disilanes and alkoxysilanes.

10. The process of claim 1 wherein said subsequent stage catalyst is selectivated ex situ by treating with a selectivating agent selected from the group consisting of polysiloxanes, siloxanes, silanes, disilanes and alkoxysilanes.

11. The process of claim 1 wherein said subsequent stage catalyst is selectivated by a process selected from the group consisting of in situ coke deposition and ex situ coke deposition.

12. The process of claim 2 wherein said first stage catalyst is selectivated by one or more treatments with a selectivating agent.

13. The process of claim 1 wherein said subsequent stage catalyst is selectivated by one or more treatments with a selectivating agent.

14. The process of claim 1 wherein said first stage catalyst comprises a silica binder.

15. The process of claim 1 wherein said subsequent stage catalyst comprises a silica binder.

16. The process of claim 1 wherein the weight ratio of the first stage catalyst to the subsequent stage catalyst ranges from >1 to 10.

17. The process of claim 1 wherein the weight ratio of the first stage catalyst to the subsequent stage catalyst ranges from 2 to 5.

18. The process of claim 1 wherein the first stage zone and the subsequent stage zone are in the same reactor vessel.

19. The process of claim 1 wherein the first stage zone and the subsequent stage zone are in separate reactor vessels.

20. A plural stage toluene conversion process for preparing xylenes comprising:
i) contacting in a first stage toluene disproportionation zone a reaction stream comprising toluene and hydrogen with a first stage catalyst which is substantially free of hydrogenation metal, said catalyst comprising HZSM-5 zeolite crystals having a major dimension of at least about 1 micron, a surface YO₂/X₂O₃ ratio which is no more than 20% greater than the bulk YO₂/X₂O₃ ratio of the crystal, and a SiO₂/Al₂O₃ ratio of 25-40, said effluent further comprising ethylbenzene;
ii) contacting said effluent from the first stage in a subsequent stage ethylbenzene abatement zone with a subsequent stage catalyst containing porous selectivated silica-bound PtZSM-5 zeolite crystals having a major dimension of at least about 1 micron, a surface SiO₂/Al₂O₃ ratio which is no more than 20% greater than the bulk SiO₂/Al₂O₃ ratio of the crystal, and a SiO₂/Al₂O₃ ratio of 25-40, which catalyst is selectivated by treating with a selectivating agent selected from the group consisting of polysiloxanes, siloxanes, silanes, disilanes and alkoxysilanes, which hinders entry of xylene isomers and permits entry of ethylbenzene into the pores of said subsequent stage catalyst molecular sieve, at reaction conditions sufficient to selectively convert said ethylbenzene to benzene and ethane in the presence of xylenes and toluene to provide a subsequent stage product containing para-xylene and having reduced ethylbenzene content relative to the effluent from said first stage.
